(19) United States
(12) Patent Application Publication
(10) Pub. No.: US 2009/0081752 A1
(43) Pub. Date: Mar. 26, 2009

(54) BIOREACTOR, KIT AND METHOD OF USING SAME

(76) Inventors: Robert G. DENNIS, Raleigh, NC (US); David A. WOLF, Houston, TX (US); Donnie RUDD, Sugar Land, TX (US)

Correspondence Address: LADAS & PARRY LLP, 224 SOUTH MICHIGAN AVENUE, SUITE 1600, CHICAGO, IL, 60604, US

(21) Appl. No.: 11/859,976

(22) Filed: Sep. 24, 2007

(51) Int. Cl. C12N 13/00; C12M 1/10; C12M 1/42

(52) U.S. Cl. 435/173.8; 435/298.2; 435/303.1

(57) ABSTRACT

A bioreactor, kit, a method of using the device to promoting growing and/or culture of a cell, and a method for regenerating and/or improving the function of mammalian cells. The bioreactor includes a controlling circuit coupled to magnetic field emitter that emits relatively steep and short-lived magnetic field pulses during these an active ephemeral period. The bioreactor also provides a relatively long-term inactive phase in which no magnetic field pulses are imposed. The kit includes the unassembled components of the bioreactor. The method of using the bioreactor and of regenerating and/or improving the function of mammalian cells includes the step of applying a time variant magnetic field through the cell to promote growing and/or culturing, and then introducing the cells to a mammal to regenerate cells.

### FIELD OF THE INVENTION

The present invention relates to cell and tissue culture systems, more particularly to a bioreactor, a kit and a method of using the bioreactor for use in regenerating mammalian cells, and a method of using the bioreactor for growing and/or culturing a cell.

### BACKGROUND

For many years there have existed methods for culturing cells. Some methods involve culturing cells in two-dimensional cultures utilizing such culture chambers as flasks and petri dishes, and others involve culturing cells in three-dimensional cultures utilizing such culture chambers as bioreactors. Methods of optimally culturing cells include adding molecules to cells in a culture such as growth factors, hormones, and others that, for instance, up or down regulate expansion of cells. Some methods are optimized for culturing individual cells and others are optimized for tissue culture. In addition, in an effort to produce more cells or larger tissue constructs over time, cell cultures are performed under various conditions.

This invention relates to a new and novel device, kit and method of using the device to improve the regeneration of cells, cell aggregates, tissue, and tissue like structures, by the induction of a sequence of electromagnetic pulses with a mandatory relaxation period between the pulses. The period of time during which the electromagnetic energy is induced, or present, in the area of interest, usually an area in need of regeneration and growth is herein referred to as the “active” period. The period of time between the active periods is referred to as the “relaxation period” also called an “inactive period.” There may be a transition period between the active and relaxation periods usually required because the apparatus and real target system does not have an instantaneous response nor is it always advisable to induce such rapid response into the target area. The preferred embodiment utilizes an extremely short active period, 200 microseconds, during which an electromagnetic field is induced over the area of a broken bone or other tissue to be regenerated. The relaxation period is preferably 100 milliseconds. The relaxation period thus can occupy 99.8% of the time. It is thought that this long relaxation period is important because during this time the tissues, surrounding interstitial components, fluids, soluble ions, and other species are able to function in a normal manner not under the influence of the electromagnetic field or pulse. However, it is also thought that the intermittent temporary presence of an electromagnetic pulse momentarily disrupts the normal tissue behavior by freeing individual species to become again mobile, by increasing their chemical activity or availability, by synchronizing their diffusive or oscillatory behavior, by changing the conformation, presentation, size or other feature of active sites, by biasing the decay rate or path or outcome of unstable species, by transient entrainment or synchronization of molecular or ionic processes, by “pinging” or “ringing” the system allowing the system to restabilize in a different preferred rest state (analogous to shaking a sieve or tapping or vibrating a container to cause the contents to settle in a different arrangement, but on a much smaller scale so the physics is different but analogous, or other transient perturbation heretofore undescribed mechanistically but resulting in observable chemical and biological effects. Collectively we refer to these mechanisms as “activation” of the species in question. These Pulse-relaxation events presumably act upon molecules and charged species directly, and the time scale of the change in the electromagnetic field is such that it corresponds to the time constants for molecular events such as ion diffusion across membranes, ligand binding and release events, altering molecule associations, and protein folding (nano-seconds to micro-seconds).

Other schemes or processes, such as bulk mechanical shaking or sinusoidal electromagnetic perturbations of low frequency do not yield similar therapeutic effects due to the physical scale of the presumed mechanisms involved, which operate at very low Reynolds number. The amplitude of the field is limited so as to facilitate these extremely rapid electromagnetic transient perturbations, but the amplitude of the field is not thought to functionally or permanently disrupt the normal activity of the species in question. It is thought that the relaxation period is a key feature of this therapeutic approach that allows the species to resume normal unbiased interactions subsequent to the imposition of the transient magnetic field pulses but provides the benefit of the new arrangement of the component species. One aspect of the invention is envisioned to impose rapidly changing magnetic fields in and around cells, cell aggregates, tissues, tissue like structures, and the like, with a second electromagnetic state. It is also possible for the active and relaxation periods to consist of different electromagnetic waveforms over time, to be repetitive or non-repetitive, and to be regular or irregular in rhythm.

Depending on the specific conditions under which the magnetic field pulse relaxation sequences are applied and the concomitant various shapes of the electromagnetic field waveforms that drives these sequences during these periods and the transition periods, different tissue function regulating effects may also be affected.

Other background conditions and therapeutic interventions are envisioned to lead to their own particular regulatory outcome but the fundamental process of activation of the species by imposing these rapidly changing alternating magnetic field pulses coupled to relatively long relaxation periods for bio-molecular processes to proceed unimpeded is a constant theme in this invention. For example, even stem cells are thought to be affected by imposing these rapidly changing alternating magnetic field pulses coupled to relatively long relaxation periods which we believe enables an enhanced interaction of the multiple species by intermittent mobilization (during the active period) and, perhaps more importantly, the provision of the relaxation period during which assimilation and organization by natural bio-molecular processes may proceed. This class of electromagnetic pulse relaxation applications may be used in combination with and enhance other treatment modalities.

### SUMMARY OF THE INVENTION

The present bioreactor, kit and method of using, according to the principles of the present invention, overcomes a number of the shortcomings of the prior art by providing a novel bioreactor, kit and method for use in promoting the growth and/or culture. The bioreactor includes a controlling circuit coupled to magnetic field emitter that emits relatively steep and short-lived magnetic field pulses during these an active ephemeral period, and a culture container located within the magnetic field generated by the magnetic field emitter. The bioreactor also provides a relatively long-term inactive phase in which no magnetic field pulses are imposed. It is thought that both these steep short-lived magnetic field pulses and the inactive periods play important roles in the growth and/or culture of cells. The kit includes the unassembled components of the bioreactor. The method of growing and/or culturing cells includes the step of applying a time variant magnetic field through the cell to promote growth and/or culture.

To attain this, the present invention essentially comprises a controlling circuit coupled to magnetic field emitter that emits relatively steep and short-lived magnetic field pulses during these an active ephemeral period in which the bioreactor also provides a relatively long-term inactive phase in which no magnetic field pulses are imposed.

There has thus been outlined, rather broadly, the more important features of the invention in order that the detailed description thereof that follows may be better understood, and in order that the present contribution of the art may be better appreciated.

The invention may also include an optional power supply.

Numerous objects, features and advantages of the present invention will be readily apparent to those of ordinary skill in the art upon reading of the following detailed description of presently preferred, but nonetheless illustrative, embodiments of the present invention when taken in conjunction with the accompanying drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an aspect of the present invention to provide a bioreactor that has many of the advantages of the prior bioreactors and minimizing a number of their disadvantages.

It is another aspect of the present invention to provide a bioreactor that may be easily and efficiently manufactured and marketed.

An even further aspect of the present invention is to provide a bioreactor that has a low cost of manufacture with regard to both materials and labor, and which accordingly is then susceptible of low prices of sale to the consuming public, thereby making the bioreactor economically available to the buying public.

Still another aspect of the present invention is to provide a bioreactor that provides in the apparatuses and methods of the prior art some of the advantages thereof, while simultaneously overcoming some of the disadvantages normally associated therewith.

Still another aspect of the present invention is to provide a kit comprising the un-interconnected elements of the bioreactor.

Lastly, it is an aspect of the present invention to provide a new and improved method of growing and/or culturing cells comprising the steps of applying a time variant magnetic field through the cells to promote growth and/or culturing of the cells.

Unless otherwise defined, all scientific and technical terms used herein are to be construed as having the same meaning as commonly understood by one of ordinary skill in the art to which this invention pertains. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present document, including definitions, will control. Unless otherwise indicated, materials, methods, and examples described herein are illustrative only and not intended to be limiting.

There has thus been outlined, rather broadly, the more important features of the invention in order that the detailed description thereof that follows may be better understood, and in order that the present contribution of the art may be better appreciated.

Numerous other features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and description matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 depicts a schematic view of a preferred embodiment of the bioreactor constructed in accordance with the principles of the present invention;

FIG. 2 depicts a perspective view of a preferred embodiment of the bioreactor;

FIGS. 3A, 3B, 3C and 3D depict a number of different embodiment configurations of the bioreactor;

FIGS. 4A, 4B, 4C, 4D, 4E and 4F depict a number of different electronic schemes of how the bioreactor can be configured;

FIGS. 5A, 5B, and 5C depict a number of electromagnetic physical characteristics experienced by the magnetic field emitter during active and inactive periods;

FIGS. 6A, 6B, 6C, 6D, 6E, 6F, and 6G depict a number of embodiments of showing different magnetic field output patterns as a function of time;

FIGS. 7A, 7B, 7C, 7D, 7E, and 7F depict various ways the bioreactor can be mounted to promote the growing and/or culturing of a cell;

FIG. 8 depicts the bioreactor internally mounted to promote growing and/or culturing of a cell;

FIG. 9 is a schematic of the electronic circuit utilized to drive the time variant magnetic field generated by the coil magnetic field emitter.

The same reference numerals refer to the same parts throughout the various figures.

### DETAILED DESCRIPTION OF THE DRAWINGS

The following detailed embodiments presented herein are for illustrative purposes. That is, these detailed embodiments are intended to be exemplary of the present invention for the purposes of providing and aiding a person skilled in the pertinent art to readily understand how to make and use of the present invention.

Accordingly, the detailed discussion herein of one or more embodiments is not intended, nor is to be construed, to limit the metes and bounds of the patent protection afforded the present invention, in which the scope of patent protection is intended to be defined by the claims and their equivalents thereof. Therefore, embodiments not specifically addressed herein, such as adaptations, variations, modifications, and equivalent arrangements, should be and are considered to be implicitly disclosed by the illustrative embodiments and claims described herein and therefore fall within the scope of the present invention.

Further, it should be understood that, although steps of various the claimed method may be shown and described as being in a sequence or temporal order, the steps of any such method are not limited to being carried out in any particular sequence or order, absent an indication otherwise. That is, the claimed method steps are to be considered to be capable of being carried out in any sequential combination or permutation order while still falling within the scope of the present invention.

Additionally, it is important to note that each term used herein refers to that which a person skilled in the relevant art would understand such term to mean based on the contextual use of such term herein. To the extent that the meaning of a term used herein, as understood by the person skilled in the relevant art based on the contextual use of such term, differs in any way from any particular dictionary definition of such term, it is intended that the meaning of the term as understood by the person skilled in the relevant art should prevail.

Furthermore, a person skilled in the art of reading claimed inventions should understand that “a” and “an” each generally denotes “at least one,” but does not exclude a plurality unless the contextual use dictates otherwise. And that the term “or” denotes “at least one of the items,” but does not exclude a plurality of items of the list.

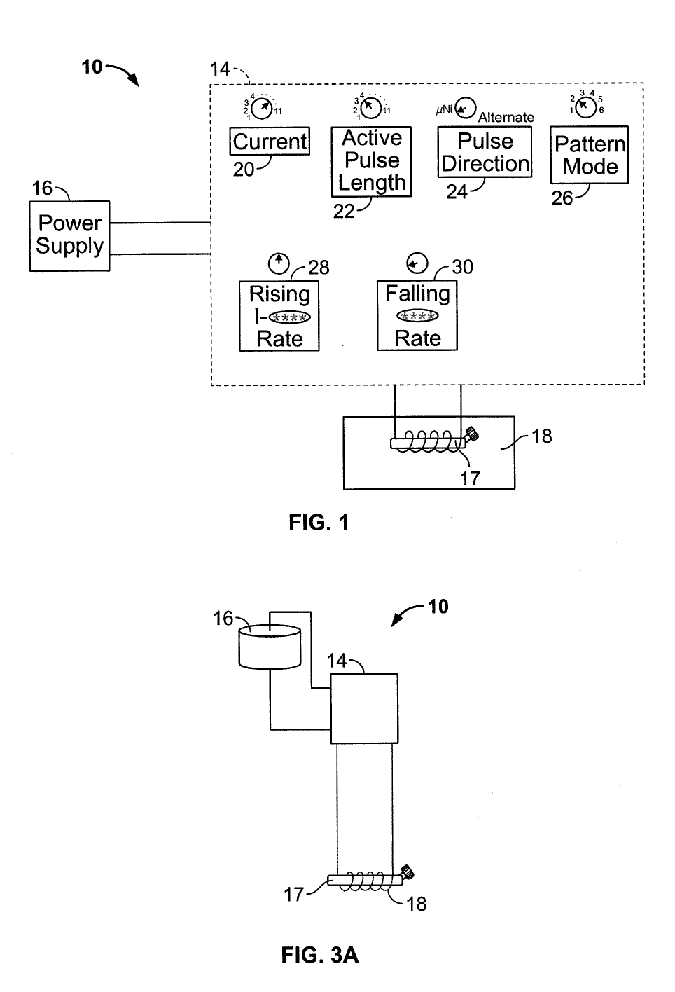

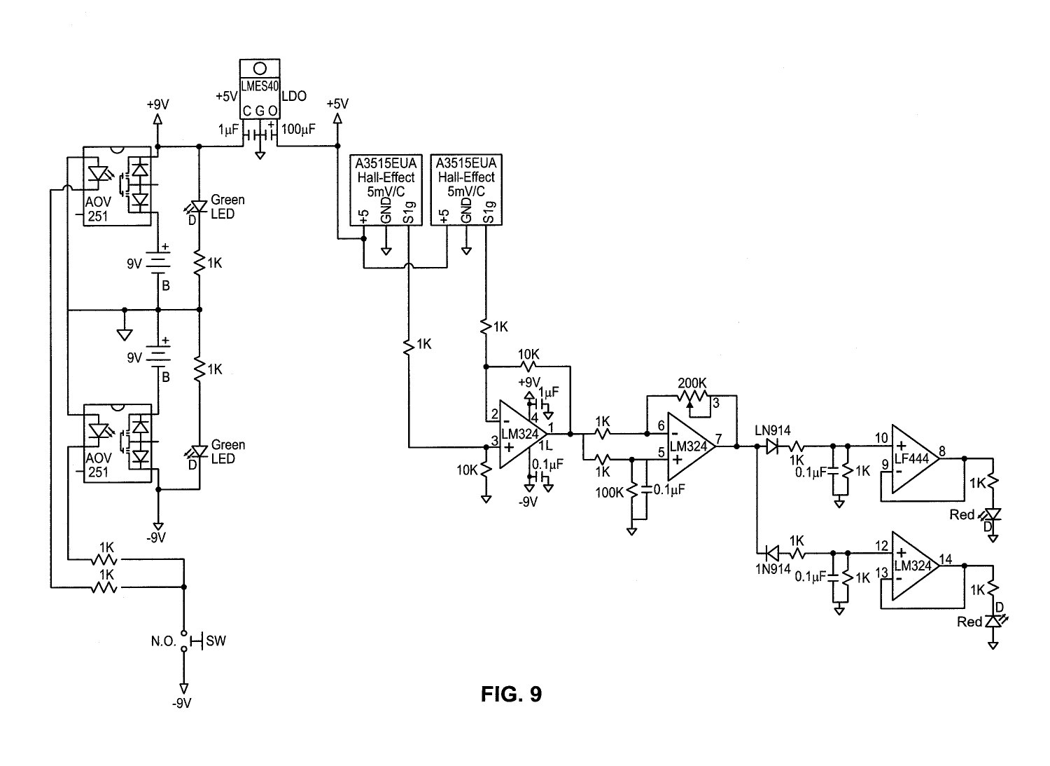

Referring now to the drawings, and in particular FIGS. 1 to 9 thereof, one preferred embodiment of the present invention is shown and generally designated by the reference numeral 10. One preferred embodiment of a bioreactor 10 for promoting the growth and/or culture of a cell comprises a controlling circuit 14, a magnetic field emitter 18, and a culture containment device 17. The controlling circuit 14 is configured to be powered by a power source 16 and is configured to output an electric pulse train. The electric pulse train outputted from the controlling circuit 14 comprises an output current, an electrical cycle period, an electrical active and inactive period, a peak voltage amplitude, and a peak current amplitude. The magnetic field emitter 18 electrically coupled to the controlling circuit 14 is configured to provide a time variant magnetic field when driven by the electric pulse train of the controlling circuit 14. The magnetic field emitter 18 electrically coupled to the controlling circuit 14 is configured to provide a time variant magnetic field when driven by the electric pulse train of the controlling circuit 14. The magnetic field emitter 18 that is electrically coupled to the controlling circuit 14 is configured to provide a time variant magnetic field comprising a magnetic (B) field exhibiting a magnetic slew rate of at least about 10 kiloGauss/sec when driven by the electric pulse train from the controlling circuit 14.

The bioreactor 10 is subject to almost any infinite number of design variations as long as the bioreactor 10 can produce a magnetic slew rate (either rising or falling, or both rising and falling) of at least about 10 kiloGauss/sec. For instance, one variation is that the magnetic field of the time variant magnetic field can be configured to exhibit a slew rate (either rising or falling, or both rising and falling) being between about 25 to about 1000 kiloGauss/sec. Another variation is that the magnetic field of the time variant magnetic field can be configured to exhibit a magnetic cycle period between about 0.01-1000 Hertz. Yet another variation is that the magnetic field of the time variant magnetic field can be configured to exhibit a magnetic field active duty between about 0.01 to 50 (preferably 0.01 to 2) percent of the cycle period wherein the magnetic active field duty defined as when the magnetic field emitter 18 emits the magnetic field. Still yet another variation is that the magnetic field of the time variant magnetic field can be configured to exhibit a magnetic inactive duty being between about 50 to 99.99 (preferably 98 to 99.99) percent of the cycle period wherein the magnetic field inactive duty defined as when the magnetic field emitter 18 does not emit the magnetic field. Even yet another variation is that the magnetic field of the time variant magnetic field can be configured to exhibit a peak magnetic amplitude being between about −20 to +20 Gauss. One variation in the design of the controlling circuit 14 is that it is configured to exhibit an electrical current slew rate (either rising or falling, or both rising and falling) between about 10 to about 1000 Amperes/sec. Another variation of the controlling circuit 14 is that the output current of the electric pulse train outputted from the controlling circuit 14 can be configured to exhibit a falling slew rate being between about 10 to about 1000 Amperes/sec. Yet another variation of the controlling circuit 14 is that it can be configured to the output the electric pulse train to exhibit an electrical cycle period being between about 0.01-100 Hertz. Still another variation of the controlling circuit 14 is that the output current of the electric pulse train outputted from the controlling circuit 14 can be configured to exhibit an electrical active period between about 0.01 to 50 (preferably 0.01 to 2) percent of the electrical cycle period wherein the electrical active period defined as when the output current is outputted. Yet another variation of the controlling circuit 14 is that the output current of the electric pulse train outputted from the controlling circuit 14 can be configured to exhibit an electrical inactive period between 50 to 99.99 preferably 98 to 99.99) percent of the electrical cycle period wherein the electrical inactive period defined as when the output current is not outputted. Still yet another variation of the controlling circuit 14 is that the output current of the electric pulse train outputted from the controlling circuit 14 can be configured to exhibit a peak voltage amplitude being between about −5 to +5 Volts and to exhibit a peak current amplitude being between about −5 to +5 kiloAmps. Even yet another variation of the controlling circuit 14 is that the electric pulse train of the controlling circuit 14 is that the output current can be configured to exhibit a rising electrical current slew rate between about 10 to about 1000 Amperes/sec and to exhibit a falling electrical slew rate being between about 10 to about 1000 Amperes/sec. Still another variation of the controlling circuit 14 is that the electrical cycle period can be configured to be between about 0.01-100 Hertz. Yet another variation of the controlling circuit 14 is that the electrical active period can be configured to be between about 0.01 to 2 percent of the electrical cycle period and that the electrical inactive period can be configured to be between 98 to 99.99 percent of the electrical cycle period. Even yet another variation of the controlling circuit 14 is that the peak voltage amplitude can be configured to be between about 1 to 10 Volts. Still yet another variation of the controlling circuit 14 is that the peak current amplitude can be configured to be being between about 1 to 10 Amps.

The magnetic field emitter 18 of the bioreactor 10 may be made of any known is selected from the group consisting of a coil magnetic field emitter 18, a plurality of coil magnetic field emitters 18, an antenna magnetic field emitter, and a plurality of loop magnetic field emitters 18. The magnetic field emitter 18 may exhibit any known inductance value.

The controlling circuit 14 of the bioreactor 10 may have an optional current switch 20 may be added to the controlling circuit 14 of the bioreactor 10 in which the optional current switch 20 is configured to control the output current of the electric pulse train outputted from the controlling circuit 14. The controlling circuit 14 of the bioreactor 10 may have an optional cycle length switch 22 may be added to the controlling circuit 14 of the bioreactor 10 in which the optional cycle length switch 22 is configured to control the electrical cycle period of the electric pulse train outputted from the controlling circuit 14. The controlling circuit 14 of the bioreactor 10 may even have an optional pulse direction switch 24 may be added to the controlling circuit 14 of the bioreactor 10 in which the optional pulse direction switch 24 is configured to control the peak voltage and current amplitudes of the electric pulse train outputted from the controlling circuit 14. The controlling circuit 14 of the bioreactor 10 may also have an optional output mode switch 26 may be added to the controlling circuit 14 of the bioreactor 10 in which the output mode switch 26 is configured to control various patterns of the electric pulse train outputted from the controlling circuit 14. Alternating polarity, or other sequences with low net DC values over time may yield the advantage of not introducing or accumulating long term net electric or magnetic motive forces. In cases where such accumulated forces are desirable, the instrumentation may be adjusted to produce such, and in a degree found to optimize the tissue response. Still yet the controlling circuit 14 of the bioreactor 10 may also have an optional rising slew rate switch 28 may be added to the controlling circuit 14 of the bioreactor 10 in which the rising slew rate switch 28 is configured to control the output current rising slew rate of the electric pulse train outputted from the controlling circuit 14. The controlling circuit 14 of the bioreactor 10 may have an optional falling slew rate switch 30 may be added to the controlling circuit 14 of the bioreactor 10 in which the falling slew rate switch 30 configured to control the output current falling slew rate of the electric pulse train outputted from the controlling circuit 14. The “slew” or rate of change of the energizing signal may be constant or variable, and in practical terms, variability is accepted in most practically realizable implementations. Variability, such as “tapering” or “wave shaping” at inflection points and sharp signal transition points are introduced or accepted as advantage in target cell response or for practical circuitry implementation.

The bioreactor 10 may optionally comprise the power source 16 electrically coupled to the controlling circuit 14. The optional power source 16 may be selected from the group consisting of a battery power source 16, a high capacity capacitor power source 16, and an electrical outlet power source 16.

The culture containment device 17 of the bioreactor 10 is in relation to the magnetic field emitter 18 such that, when in use a magnetic field is generated by the magnetic field emitter 18, the magnetic field affects the cells in the culture container. By the term “in relation to” it is intended that the culture containment device 17 is located a preferred distance from the magnetic field emitter, including, but not limited to, within the magnetic field emitter which would be a distance of zero, such that in use, the magnetic field generated by the magnetic field emitter 18 reaches the interior portion of the culture containment device 17 which is adapted to receive cells. By the term “cells” it is intended throughout the disclosure herein that all type of cell be encompassed thereby including, but not limited to, a cell, a cell aggregate, a tissue, a tissue construct, a tissue like structure, and the same. A “culture containment device” it is intended to comprise any and all vessels adapted to receive and supporting a cell culture. Furthermore, the culture containment device 17 may be a single culture container or more than one culture container, as preferred. The culture containment device 17 may be any preferred configuration that can be functionally located within the magnetic field generated by the magnetic field emitter 18 when the bioreactor 10 is in use.

One preferred embodiment of a kit for a bioreactor 10 comprises a magnetic field emitter 18 coupled to a controlling circuit 14. The magnetic field emitter 18 is configured to be electrically coupled to the controlling circuit 14 in which the magnetic field emitter 18 is configured to provide a time variant magnetic field when driven by the electric pulse train of the controlling circuit 14. The time variant magnetic field comprises a magnetic (B) field exhibiting a magnetic slew rate of at least about 10 kiloGauss/sec. The controlling circuit 14 may be configured to be powered by a power source 16 and is also configured to output an electric pulse train.

The controlling circuit 14 of the kit of the bioreactor 10 may optionally have a current switch 20 which is configured to control the electrical cycle period of the electric pulse train to output from the controlling circuit 14. The controlling circuit 14 of the kit of the bioreactor 10 may also optionally have a cycle length switch 22 configured to control the electrical cycle period of the electric pulse train outputted from the controlling circuit 14. The controlling circuit 14 of the kit of the bioreactor 10 may also optionally have a pulse direction switch 24 configured to control the peak voltage and current amplitudes of the electric pulse train outputted from the controlling circuit 14. The controlling circuit 14 of the kit of the bioreactor 10 may also optionally have an output mode switch 26 configured to control various patterns of the electric pulse train outputted from the controlling circuit 14. The controlling circuit 14 of the kit of the bioreactor 10 may also optionally have a rising slew rate switch 28 configured to control the output current rising slew rate of the electric pulse train outputted from the controlling circuit 14. The controlling circuit 14 of the kit of the bioreactor 10 may also optionally have a falling slew rate switch 30 configured to control the output current falling slew rate of the electric pulse train outputted from the controlling circuit 14.

The magnetic field emitter 18 of the kit of the bioreactor 10 may be any known commercially available magnetic field emitter 18. Some preferred magnetic field emitters 18 may be selected from the group consisting of a coil magnetic field emitter 18, a plurality of coil magnetic field emitters 18, a plurality of loop magnetic field emitters 18, and an antenna magnetic field emitter 18.

The kit of the bioreactor 10 also comprises a culture containment device 17. The culture containment device 17 is preferably configured to the intended test, result, and/or cell to be grown and/or cultured. A preferred configuration of the culture containment device 17 is a rotating bioreactor, rotatable about a substantially horizontal longitudinal central axis. Another preferred configuration of the culture containment device 17 is a petri dish, more preferably a flask, and most preferably a plate.

An optional power source 16 may be added to the kit of the bioreactor 10 in which the optional power source is configured to be electrically coupled to the controlling circuit 14. The power source 16 of the kit of the bioreactor 10 may be any known power source 16 in which some preferred power sources 16 may be selected from the group consisting of a battery, a high capacity capacitor, and an electrical outlet.

An optional stabilizing agent 32 may be added to the kit of the bioreactor 10 in which the stabilizing agent 32 may be any known and commercially available stabilizing agent 32. Preferably, the stabilizing agent 32 is made from a non-conductive material. The stabilizing agent provides support and positioning to the magnetic field emitter 18 such that, in use, the bioreactor 10 can function to provide a magnetic field in the interior portion of the culture containment device 17 and to the cells contained therein.

One preferred method for growing and/or culturing a cell comprises the step of applying a time variant magnetic field through the cell to promote the growth and/or culture of the cell. By the term “growing and/or culturing” and similar terms, it is intended that throughout this paper, either a cell is increased in number to more than one or more than the number that was present in the culture containment device before the bioreactor was in use (growth) and/or the cell is merely exposed to the time variant magnetic field generated by the magnetic field emitter 18, in which case the number of cells in the culture containment device may increase, decrease, or remain unchanged. A combination of the growth and culture may also be preferred. Preferably, the desired outcome and final use of the cells will dictate the preferred method whether it be growing the cells in number, culturing the cells, or growing and/or culturing the cells. Therefore, by the term “desired level” it is intended that the cell, cell aggregate, tissue, and/or tissue like structure, by the method of the present invention grow and/or culture to a level that is most beneficial for the object of the culture. For instance, if in a preferred embodiment the object of the culture is to achieve a greater number cells Y than the original number before growing and/or culturing X, then as soon as the cells reach Y, then the desired level is reached. In another preferred embodiment, if the object of the culture is merely culturing the cells until certain membrane proteins are up regulated and others down regulated by a factor of X or Y, then once the membrane proteins are detected at that level, regardless of the number of cell, then the desired level has been reached.

It is expected that growing and/or culturing cells by this method may provide the cells with a unique phenotypic expression not possible by any other method. The unique phenotypic expression of a cell can be ascertained by any method well known in the art including Affymetrix Gene Array Data and functional assays. It is well known in the art that the phenotypic expression of a cell can be determined by detecting transcribed RNA patterns (RNA phenotype) coding for such proteins as multiple cell structural and functional proteins, and also cell sub organelles, but not limited thereto. Cell structural proteins may include cell membrane components and cell cytoskeletal components, and cell functional proteins may, among others, effect cell metabolism. Depending on the cell, some genes may be up regulated by the time variant magnetic field, and others may be down regulated. Not to be bound by theory, but cells cultured by this method may have heightened functionality. Also, not to be bound by theory, but cells cultured by this method produce significantly different cells with significantly distinct expressions from cells grown by any other method.

In the preferred embodiment wherein the growing and/or culturing a cell comprises the step of applying a time variant magnetic field through the cell, the applying step comprises a magnetic field having a slew rate, either a rising or a falling, or both a rising and falling) of at least 10 kiloGauss/sec.

The applying step may last for any known length of time in which one preferred embodiment is that the applying step is applied for a duration of at least one week without interruption. Another preferred embodiment is that the applying step lasts for a duration of at least one week and is performed at least 8 hours in each day during the duration of the applying step.

The time variant magnetic field may be applied in any known direction. One preferred embodiment is that the time variant magnetic field is always applied along a substantially identical direction during the applying step, whereby the time variant magnetic field being a unidirectional time variant magnetic field. Another preferred embodiment is that the time variant magnetic field is alternately applied along substantially alternate opposite directions during the applying step, whereby the time variant magnetic field being an alternating bi-directional time variant magnetic field.

Another embodiment is that the time variant magnetic field is applied using a current pulse train through a magnetic field emitter generated by a circuit. A more preferred the time variant magnetic field comprises the active duty is between about 0.1 to about 1 percent of the cycle period; the rising edge magnetic slew of at least about 10 kiloGauss/sec; and the falling edge magnetic slew rate being of at least about 10 kiloGauss/sec. One preferred embodiment of the electric pulse train comprises an output current exhibiting a rising slew rate between of at least 1 Amperes/sec; the output current exhibiting a falling slew rate of at least about 1 Amperes/sec; an electrical cycle period being at least about 0.01 Hertz; an electrical active periodicity may be any function, such as being between about 0.01 to 2 percent of the electrical cycle period wherein the electrical active period defined as when the output current is outputted; an electrical inactive period between 98 to 99.99 percent of the electrical cycle period wherein the electrical inactive period defined as when the output current is not outputted; a peak voltage amplitude being between about −5 to +5 Volts; and a peak current amplitude being between about −5 to +5 kiloAmps.

It is envisioned that the present method is suitable for promoting the growing and/or culturing of a cell, preferably an animal cell, more preferably a mammalian cell. Preferably, the mammalian cell is selected from the group consisting of human, goat, sheep, domesticated dog, domesticated cat, a rat, mouse, guinea pig, rabbit, horse, cow, llama, alpaca, mule, donkey, gorilla, gibbon, orangutan, chimpanzee, lemur, rhinoceros, monkey, bat, bison, camel, wolf, coyote, fox, jackal, tiger, oryx, water buffalo, elephant, giraffe, antelope, deer, elk, lion, cheetah, panda, leopard, puma, serval, opossum, kangaroo, platypus, armadillo, lemur, muskox, baboon, zebra, pig, koala, tasmanian devil, manatee, and wombat.

It is envisioned that the present method is suitable for regenerating and/or improving the function(s) of cells in mammals selected from the group consisting of a human, a goat, a sheep, a domesticated dog, a domesticated cat, a rat, a mouse, a guinea pig, a rabbit, a horse, a cow, a llama, an alpaca, a mule, a donkey, a gorilla, a chimpanzee, a lemur, a rhinoceros, a monkey, a bat, a bison, a camel, a wolf, a coyote, a fox, a jackal, tiger, an oryx, a water buffalo, a elephant, a giraffe, an antelope, a deer, an elk, a lion, a cheetah, a panda, a leopard, a puma, a serval, an opossum, a kangaroo, a platypus, an armadillo, a lemur, a muskox, a baboon, a zebra, a pig, a koala, a tasmanian devil, a manatee, and a wombat. It expressly includes companion animals (such as dogs, cats, rabbits and the like) farm and working animals (such as horses, cows and the like) and laboratory animals.

An optional aligning step may be added to the method in which the aligning step is used to align the cell in a desired orientation.

An optional stabilizing step may be added to the method in which the stabilizing step is used to stabilize the magnetic field emitter 18 by providing support with a stabilizing agent 32.

An optional mounting step may be added to the method in which the mounting step is used to mount a magnetic field emitter 18 near a culture containment device 17. The mounting step of the magnetic field emitter 18 may be performed in any known manner such as being mounted external relative to the culture containment device 17, or may be mounted such that the culture containment device 17 in contained within the magnetic field emitter 18. If the magnetic field emitter 18 is mounted external to the culture containment device 17 it is considered to be “adjacent to” the culture containment device 17. By the term “adjacent” it is meant that, when in use, the magnetic field emitter 18 applies a magnetic field to the cells in the culture containment device 17. When the magnetic field emitter 18 contains the culture containment device 17 within, it is referred to by the term “around” wherein it is intended that the magnetic field emitter 18 encompass the culture containment device 17, so that in use, the cells in the culture containment device 17 are exposed to a magnetic field generated by the magnetic field emitter, preferably a uniform magnetic field, preferably uniformly exposed.

An optional turning off step may be added to the method in which the turning off step is used to turn off the time variant magnetic field after a substantial amount of growing and/or culturing has occurred.

An optional withdrawing step may be added to the method in which the withdrawing step is used to withdraw the magnetic field emitter 18 away from the culture containment device 17 subsequent to when the growing and/or culturing of the cells has occurred.

An optional stabilizing step may be added to the method in which the stabilizing step is used to stabilize the cells subsequent to when the cell has grown and/or cultured. The stabilizing agent 32 may be any known stabilizing agent 32. The stabilizing agent 32 may preferably be a device for supporting the magnetic field emitter in a preferred configuration. The stabilizing agent 32 may preferably provide support to the magnetic field emitter, and may also preferably position the magnetic field emitter 18 a distance from the cell to be grown and/or cultured, preferably in a culture containment device 17. The distance may preferably be zero if the culture containment device 17 is within the magnetic field emitter 18. The distance from the culture containment device 17 is such that when in use, the culture containment device 17 is functionally located within the magnetic field generated by the magnetic field emitter 18. In a preferred embodiment the culture containment device 17 is located adjacent to the magnetic field emitter 18. In such an embodiment, the magnetic field emitter 18 is functionally located adjacent to the culture containment device 17, wherein in use, the magnetic field generated by the magnetic field emitter 18 is applied to the cell in the culture containment device 17.

Referring now to FIG. 1 which depicts a schematic view of a preferred embodiment of the bioreactor 10 showing the optional power supply 16 electrically coupled to the controlling circuit 14, and the culture containment device 17. The controlling circuit 14 is shown having the optional current switch 20, the cycle length switch 22, the pulse direction switch 24, the output mode switch 26, the rising slew rate switch 28, the falling slew rate switch 30. Also shown is the magnetic field emitter 18 electrically coupled to the controlling circuit 14.

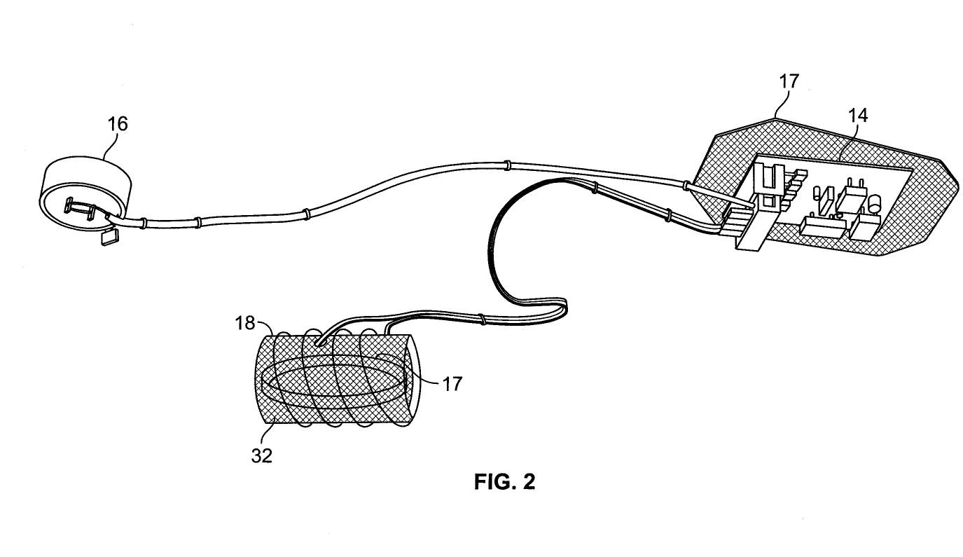

Referring now to FIG. 2 which depicts a perspective view of a preferred embodiment of the bioreactor 10 showing the assembly of an optional power supply 16 and the magnetic field emitter 18 electrically coupled to the controlling circuit 14, to a culture containment device 17 which, upon assembly, will be functionally located within the magnetic field emitter 18. FIG. 2 also depicts a stabilizing agent 32. In this embodiment, the stabilizing agent 32 provides support to the magnetic field emitter 18 such that, in use, it remains in relation to the culture containment device 17 so that a magnetic field is generated within the culture containment device 17 and applied to the cell therein.

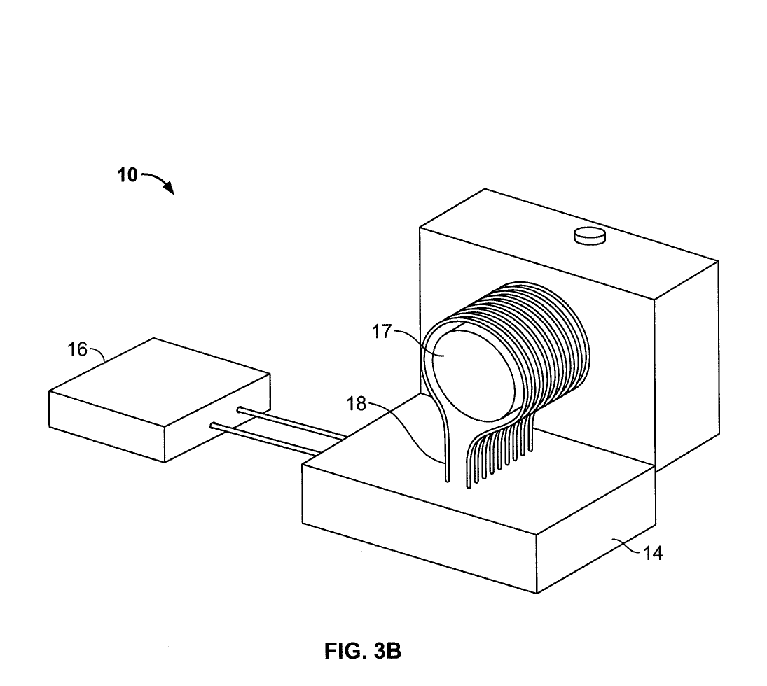

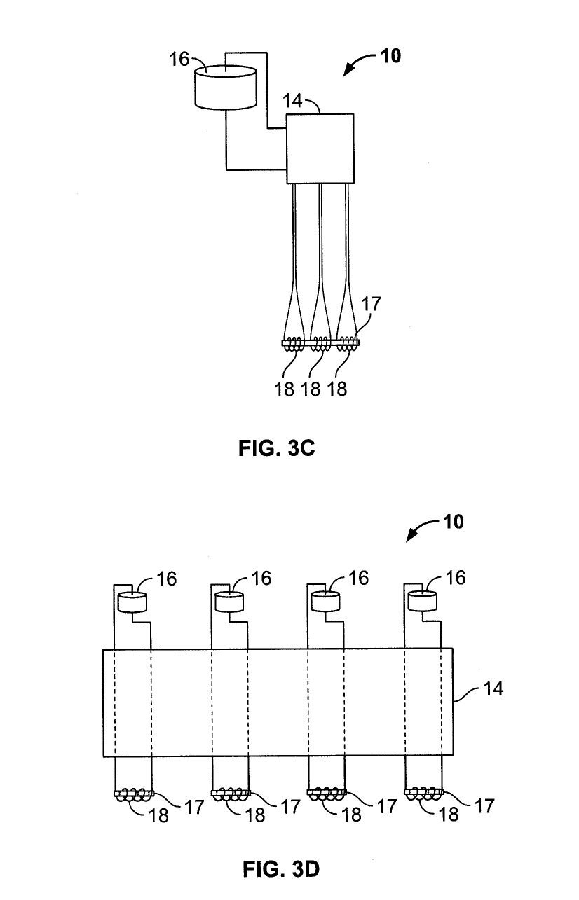

Referring now to FIGS. 3A, 3B, and 3C which depict a number of different embodiments of the bioreactor 10. The bioreactor 10 is shown having any number of different designs or configurations. FIG. 3A illustrates one preferred configuration of the controlling circuit 14 which is coupled to only one coil magnetic field emitter 18 and is powered by only one power supply 16, and a culture containment device 17, preferably a flask. FIG. 3B illustrates another preferred configuration of the controlling circuit 14 which is coupled to a plurality of loop magnetic field emitters 18 and is powered by only one power supply 16, and a culture containment device 17 is preferably a rotatable bioreactor. FIG. 3B also depicts a stabilizing agent 32. FIG. 3C illustrates yet another preferred configuration of the controlling circuit 14 which is coupled to a plurality of coil magnetic field emitters 18 and is powered by only one power supply 16, and a culture containment device 17, preferably a petri dish. FIG. 3D illustrates still yet another preferred configuration of the controlling circuit 14 which is coupled to a plurality of coil magnetic field emitters 18 and is coupled to a plurality of power supplies 16, and a culture containment device 17, preferably a plate. In FIG. 3D each power supply 16 is shown configured via the controlling circuit 14 to individually drive only a single corresponding coil magnetic field emitters 18.

A bioreactor 10 that is preferably a rotatable bioreactor is rotatable about a substantially horizontal axis, 360° in one direction, so that in use, the cells are suspended in an essentially quiescent three-dimensional environment that provides for low shear stress and turbulence. Such a three-dimensional environment also provides that cells grown and/or cultured therein have a unique phenotypic expression due to the cells adaptation to the environment in the rotatable bioreactor. The rotation of a rotatable bioreactor provides a stabilized culture environment into which cells may be introduced, suspended, maintained, grown, cultured and/or expanded with improved retention of delicate three-dimensional structural integrity by simultaneously minimizing the fluid shear stress, providing three-dimensional freedom for cell and substrate spatial orientation, and increasing localization of cells in a particular spatial region for the duration of the expansion. It is expected that rotation along with exposure to a time variant magnetic field provides the cells that are grown and/or cultured in the bioreactor 10 of the present invention with a genetic expression that is unique.

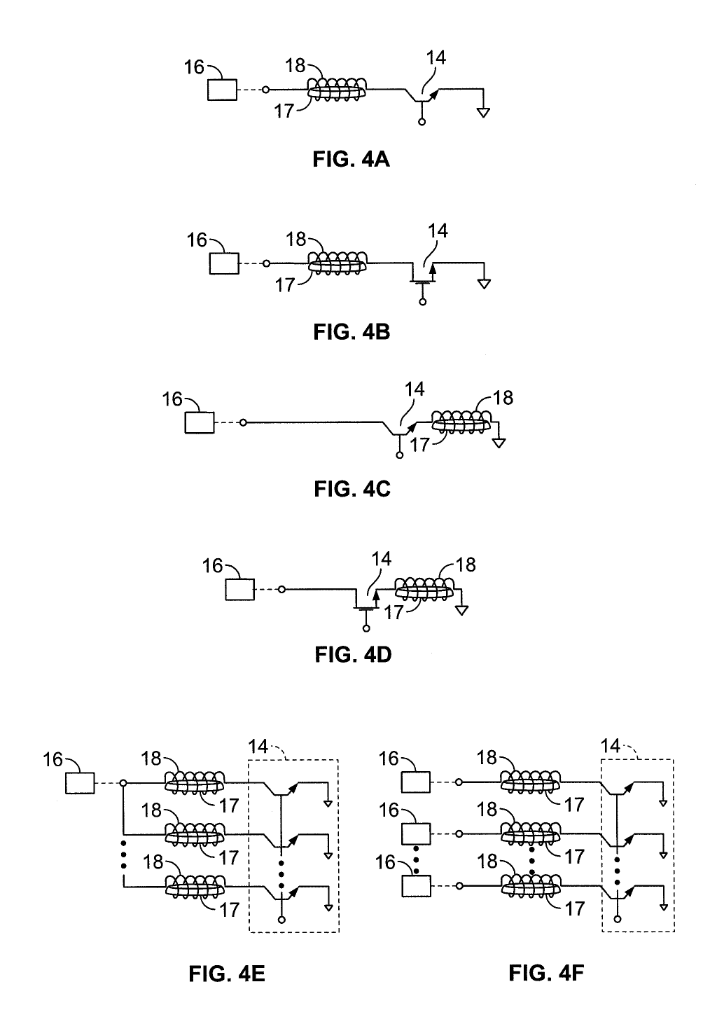

Referring now to FIGS. 4A, 4B, 4C, 4D, 4E, and 4F which depict a number of different electronic schemes of how the bioreactor 10 can be configured. These electronic schemes are depicted to illustrate just a few of the infinite number of electronic configurations of the bioreactor 10 as long as each can realize the invention as described in the claims.

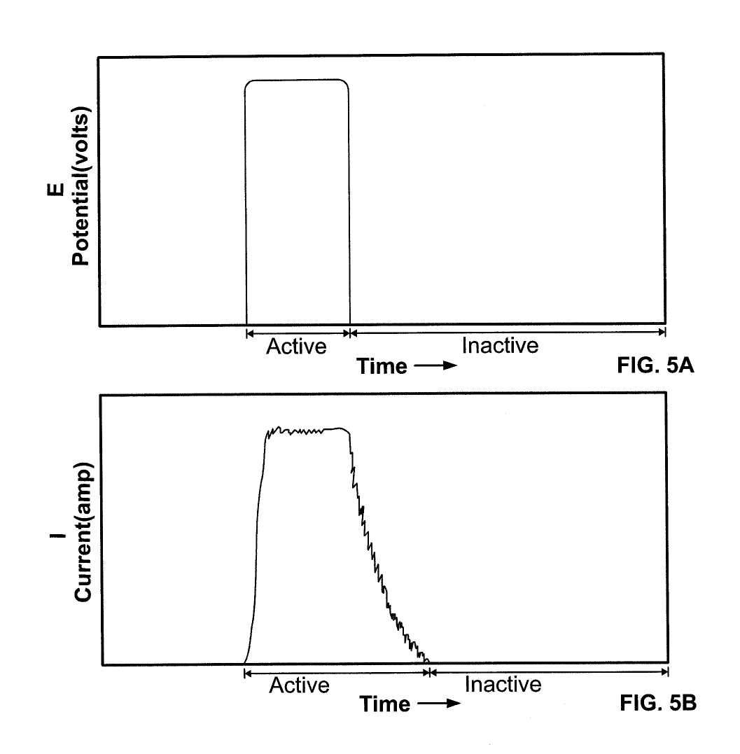

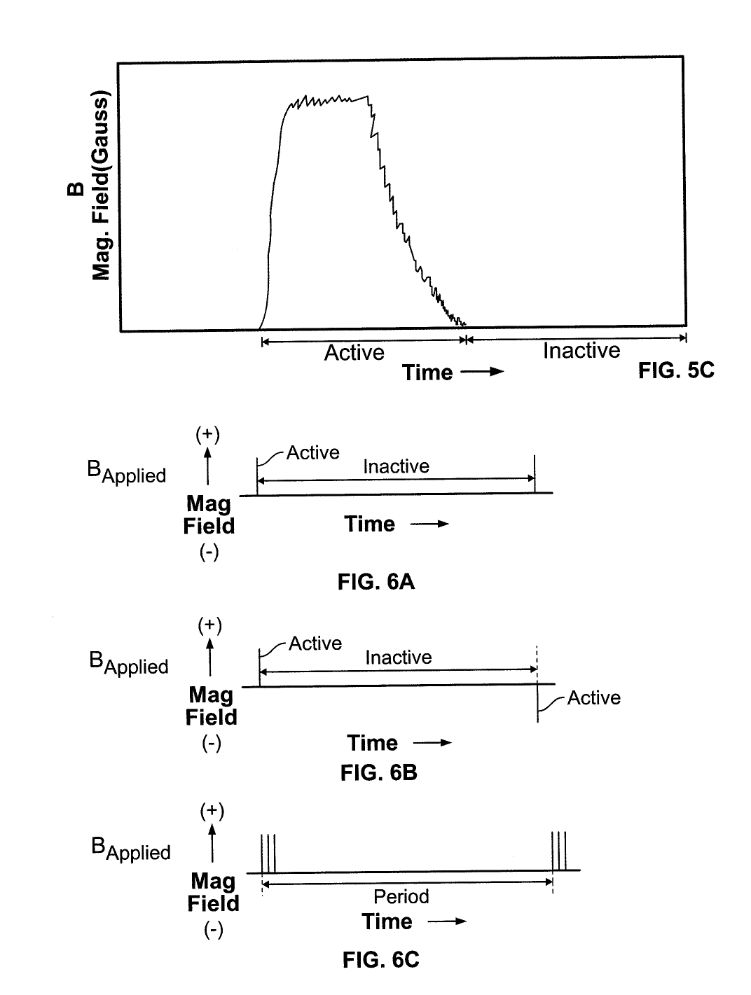

Referring now to FIGS. 5A, 5B, and 5C which depict a number of electromagnetic physical characteristics experienced by the magnetic field emitter 18 during active and inactive periods. FIG. 5A depicts a voltage step function across the magnetic field emitter 18 showing an almost instantaneous potential jump between two potential states (i.e., on state and off state). FIG. 5B depicts a current step function across the magnetic field emitter 18 showing an out of phase or delayed current, relative to the voltage step function, through the magnetic field emitter 18. FIG. 5C depicts a magnetic field step function emitted from the magnetic field emitter 18 showing an out of phase or delayed magnetic field, relative to the voltage step function, in which the magnetic field step function is approximately in phase with the current step function.

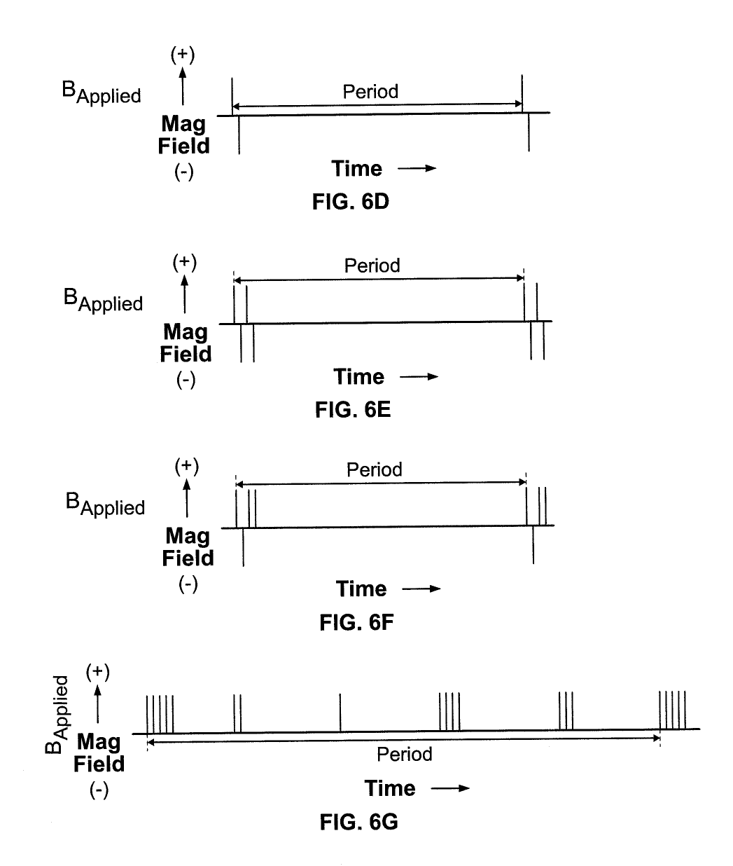

Referring now to FIGS. 6A, 6B, 6C, 6D, 6E, 6F, and 6G which depict a number of embodiments of showing different magnetic field output patterns as a function of time. The magnetic field emitter 18 is envisioned to be capable of producing any number of different patterns or modes of the resultant magnetic field along with being capable of producing alternating directional magnetic fields. As shown in FIG. 6A, one embodiment of the bioreactor 10 provides that the magnetic field emitter 18 driven by the controlling circuit 14 can be configured to produce a unidirectional magnetic field for a short time period (i.e., during the active mode) and afterwards remain quiescent (i.e., the inactive mode) until the end of the cycle period. As depicted in FIG. 6B another embodiment of the bioreactor 10 provides that the magnetic field emitter 18 driven by the controlling circuit 14 can be configured to produce alternately produce magnetic field pulses in opposite directions. Accordingly, the bioreactor 10 is envisioned to be capable of producing the various magnetic field pulse patterns as depicted in FIGS. 6A, 6B, 6C, 6D, 6E, 6F, and 6G. which are illustrative and not limited to the infinite number of magnetic field pulse patterns that the bioreactor 10 is envisioned to be capable of producing.

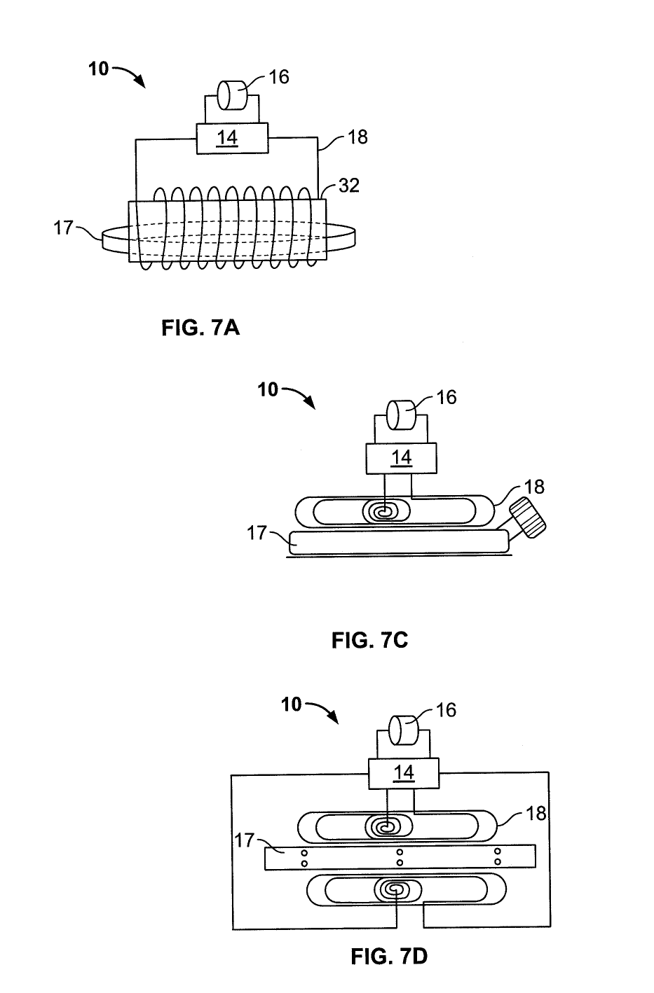

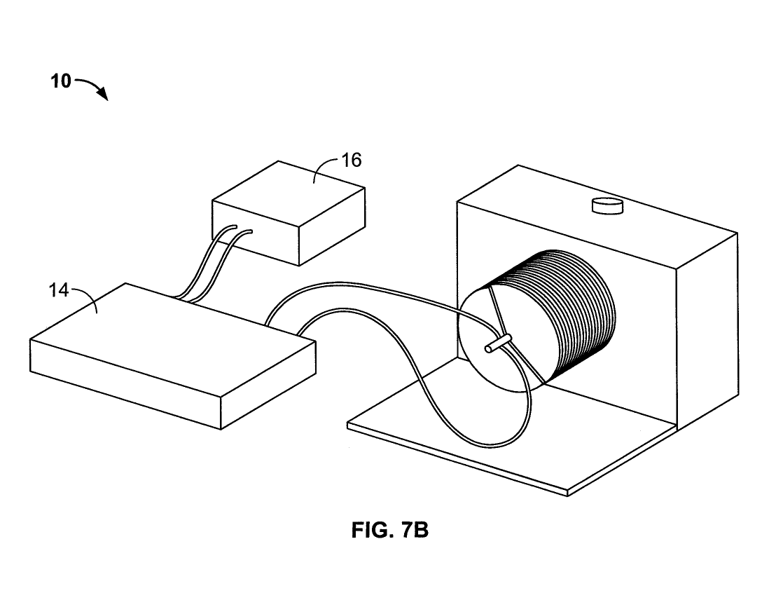

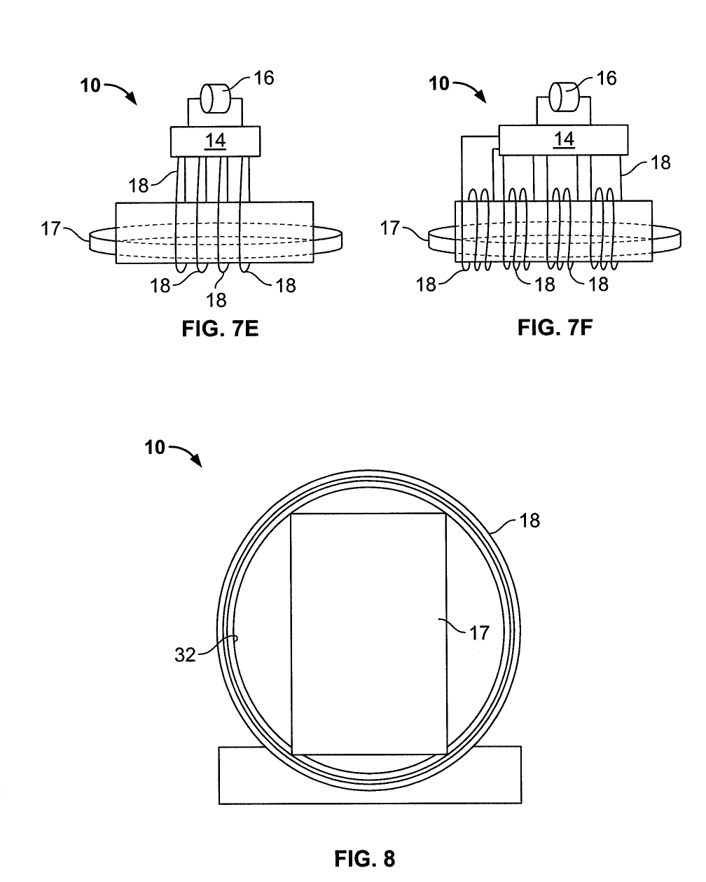

Referring now to FIGS. 7A, 7B, 7C, 7D, 7E, and 7F which depict various ways the bioreactor 10 can be externally mounted to promote growing and/or culturing of a cell. Each figure, (i.e., FIGS. 7A, 7B, 7C, 7D, 7E, and 7F) shows the controlling circuit 14 operationally coupled to the optional power source 16 and operationally coupled to at least one magnetic field emitter 18. FIG. 7B depicts that the magnetic field emitter 18 can be mounted within a stabilizing agent 32.

Referring now to FIG. 8, FIG. 8 shows the controlling circuit 14 operationally coupled to the optional power source 16 and operationally coupled to a coil magnetic field emitter 18 with a culture containment device 17 functionally located within the magnetic field emitter 18. Also shown in FIG. 8 is the stabilizing agent 32.

As to the manner of usage and operation of the present invention, the same should be apparent from the above description. Accordingly, no further discussion relating to the manner of usage and operation will be provided.

While a preferred embodiment of the bioreactor has been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Throughout this specification, unless the context requires otherwise, the word “comprise” or variations such as “comprises” or “comprising” or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modification which fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A bioreactor for growing and/or culturing cells said bioreactor comprising:
a magnetic field emitter electrically coupled to a controlling circuit, the magnetic field emitter configured to provide a time variant magnetic field when driven by an electric pulse train from the controlling circuit, the time variant magnetic field comprising a magnetic (B) field exhibiting a peak slew rate of at least about 10 kiloGauss/sec; and
the controlling circuit electrically coupled to the magnetic field emitter wherein the controlling circuit is configured to be powered by a power source and the controlling circuit is configured to output an electric pulse train driving the magnetic field emitter.

2. A bioreactor as in claim 1 wherein the B field is induced secondary to a time varying electric field.

3. A bioreactor as in claim 1 wherein the B field is the magnetic field component of an emitted electromagnetic field.

4. A bioreactor as in claim 1 wherein the time varying magnetic field is generated by current through a conductor.

5. A bioreactor as in claim 2 wherein the time varying electric field is generated by a voltage across separated conductors.

6. A bioreactor as in claim 3 wherein the electromagnetic field is emitted by an antenna magnetic field emitter carrying a time varying current.

7. The bioreactor of claim 1 wherein the magnetic slew rate is a rising magnetic slew rate.

8. The bioreactor of claim 1 wherein the magnetic slew rate is a falling magnetic slew rate.

9. The bioreactor of claim 1 further comprising the power source electrically coupled to the controlling circuit.

10. The bioreactor of claim 9 further comprising the power source which is selected from the group consisting of a battery power source, a high capacity capacitor power source, and an electrical outlet power source.

11. The bioreactor of claim 1 wherein the magnetic field emitter is selected from the group consisting of a coil magnetic field emitter, a plurality of coil magnetic field emitters, a single loop magnetic field emitter, and an antenna magnetic field emitter.

12. The bioreactor of claim 1 wherein the bioreactor is a rotatable bioreactor rotatable about a substantially horizontal axis.

13. A bioreactor kit, said kit comprising:
a magnetic field emitter electrically coupleable to a controlling circuit, the magnetic field emitter configured to provide a time variant magnetic field when driven by an electric pulse train from the controlling circuit, the time variant magnetic field comprising a magnetic (B) field exhibiting a maximum slew rate of at least about 10 kiloGauss/sec; and
the controlling circuit electrically coupleable to the magnetic field emitter, wherein the controlling circuit configured to be powered by a power source, the controlling circuit configured to output the electric pulse train driving the magnetic field emitter.

14. The kit of claim 13 further comprising the power source configured to be electrically coupled to the controlling circuit.

15. The kit of claim 13 further comprising a stabilizing agent.

16. The kit of claim 15 wherein the stabilizing agent is a device for holding the magnetic field emitter in close proximity to the culture containment device.

17. The kit of claim 13 wherein the magnetic field emitter is selected from the group consisting of a coil magnetic field emitter, a plurality of coil magnetic field emitters, a loop magnetic field emitter, a plurality of loop magnetic field emitters, and an antenna magnetic field emitter.

18. The kit of claim 13 further comprising the power source which is selected from the group consisting of a battery, a high capacity capacitor, and an electrical outlet.

19. A method for growing and/or culturing a cell, said method comprising the step of applying a time variant magnetic field through the cell to promote growing and/or culturing of the cell, wherein the time variant magnetic field comprises:
a slew rate of at least about 10 kiloGauss/sec;
a magnetic cycle period of at least about 0.01 Hertz;
a magnetic field active duty of at least about 0.01 percent of the cycle period wherein the magnetic active field duty defined as when the magnetic field emitter emits the magnetic field;
and a peak magnetic amplitude having an absolute value of at least about 0.1 Gauss; and
the controlling circuit configured to be electrically coupled to the magnetic field emitter and the controlling circuit configured to be powered by a power source, the controlling circuit configured to output an electric pulse train driving the magnetic field emitter.

20. The method of claim 19 wherein the applying step is applied from the group of durations consisting of a duration of at least one week without interruption and a duration of at least one week and being performed at least 8 hours in each day during the duration of the applying step.

21. The method of claim 19 wherein the magnetic field emitter is selected from the group consisting of a coil magnetic field emitter, a plurality of coil magnetic field emitters, at least one loop magnetic field emitter, and an antenna magnetic field emitter.

22. The method of claim 19 wherein the mounting of the magnetic field emitter is around the cell.

23. The method of claim 19 wherein the mounting of the magnetic field emitter is adjacent to the cell.

24. The method of claim 19 wherein the cell is an animal cell.

25. A method for regenerating and/or improving the functionality of mammalian cells, said method comprising the step of applying a time variant magnetic field through a mammalian cell to promote growing and/or culturing of the cell and thereafter introducing the cell into a mammal to regenerate and/or improve in function cells within the mammal, wherein the time variant magnetic field comprises:
a slew rate of at least about 10 kilo Gauss/sec; a magnetic cycle period of at least about 0.01 Hertz;
a magnetic field active duty of at least about 0.01 percent of the cycle period wherein the magnetic active field duty defined as when the magnetic field emitter emits the magnetic field; and
a peak magnetic amplitude having an absolute value of at least about 0.1 Gauss; and the controlling circuit configured to be electrically coupled to the magnetic field emitter and the controlling circuit configured to be powered by a power source, the controlling circuit configured to output an electric pulse train driving the magnetic field emitter.
